SEQUENCE LISTING (17 sequences)

SEQ ID NO: 1 - protein, 51 aa, Homo sapiens
LHLWLSGEPVQSSGTKDMRSKSDSKRVSDKQLISKAVWWTFFLPSTLWERK

SEQ ID NO: 2 - DNA, 153 bp, Homo sapiens
catctctggcttagtggggagccagtccagagctctggaacaaaggacatgagatccaaatccgattccaagcgagtgagtgacaagcagctaatttccaaagctgtgtggtggacattttttcttccttcaaccctctgggagagaaaatga

SEQ ID NO: 3 - DNA, 255 bp, Homo sapiens
atgatggcactcagaagccaggggctcatgttaccccagagctgcccacaactggctttcctcaccctaagtgccttggcagcagtgtctttttcagctctgcatctctggcttagtggggagccagtccagagctctggaacaaaggacatgagatccaaatccgattccaagcgagtgagtgacaagcagctaatttccaaagctgtgtggtggacattttttcttccttcaaccctctgggagagaaaatga

SEQ ID NO: 4 - protein, 84 aa, Homo sapiens
MMALRSQGLMLPQSCPQLAFLTLSALAAVSFSALHLWLSGEPVQSSGTKDMRSKSDSKRVSDKQLISKAVWWTFFLPSTLWERK

SEQ ID NO: 5 - protein, 13 aa, Homo sapiens
WTFFLPSTLWERK

SEQ ID NO: 6 - protein, 23 aa, Homo sapiens
SGEPVQSSGTKDMRSKSDSKRVS

SEQ ID NO: 7 - protein, 23 aa, Homo sapiens
DKQLISKAVWWTFFLPSTLWERK

SEQ ID NO: 8 - protein, 8 aa, Homo sapiens
PSTLWERK

SEQ ID NO: 9 - protein, 13 aa, Homo sapiens
AVWWTFFLPSTLW

SEQ ID NO: 10 - protein, 16 aa, Homo sapiens
KREWLTSPLFFTWWVA

SEQ ID NO: 11 - protein, 5 aa, Homo sapiens
WTFFL

SEQ ID NO: 12 - protein, 56 aa, Mus sp.
MDLSIRLSLACWELNQVSGAWGMSLKSHFKFMSDKQLISKAVQRIFFSPSTLWGEK

SEQ ID NO: 13 - protein, 34 aa, Mus sp.
MSLKSHFKFMSDKQLISKAVQRIFFSPSTLWGEK

SEQ ID NO: 14 - protein, 56 aa, artificial sequence
  analogue of Seq. ID No. 12
MDLSIRLSLACWELNQVSGAWGMSLKSHFKFMSDKQLISKAVXXXFFSPSTLWXXK

SEQ ID NO: 15 - protein, 34 aa, artificial sequence
  analogue of Seq. ID No. 13
MSLKSHFKFMSDKQLISKAVXXXFFSPSTLWXXK

SEQ ID NO: 16 - DNA, 171 bp, Mus sp.
atggacctttccatccgtctgtctcttgcttgctgggagctgaaccaggtctctggagcatggggcatgagcttaaaatcccatttcaagttcatgagtgacaagcagctaatttccaaagctgtgcagcggatatttttttctccttcaaccctctggggggaaaaatga

SEQ ID NO: 17 - DNA, 105 bp, Mus sp.
atgagcttaaaatcccatttcaagttcatgagtgacaagcagctaatttccaaagctgtgcagcggatatttttttctccttcaaccctctggggggaaaaatga

(19) United States
(12) Patent Application Publication
(10) Pub. No.: US 2010/0204097 A1
(43) Pub. Date: Aug. 12, 2010

(54) THERAPEUTIC METHODS USING A THYMUS PEPTIDE

(75) Inventors: Yoram Devary, Jerusalem (IL); Uziel Sandler, Jerusalem (IL)

Correspondence Address: THE NATH LAW GROUP, 112 South West Street, Alexandria, VA, 22314, US

(73) Assignee: IMMUNE SYSTEM KEY LTD., Jerusalem (IL)

(21) Appl. No.: 12/448,314

(22) PCT Filed: Dec. 18, 2007
(86) PCT No.: PCT/IL07/01563

§ 371 Date: Jun. 17, 2009

Related U.S. Application Data
(60) Provisional application No. 60/875,366, filed on Dec. 18, 2006.

(51) Int. Cl. A61K 38/16; A61K 38/08; C07K 14/00; C07H 21/04; C07K 16/18; A61P 9/10

(52) U.S. Cl. 514/12; 514/16; 530/324; 536/23.1; 530/389.1

(57) ABSTRACT

A method for treating or preventing a disease involving a cell having a T1/ST2 receptor, including administering to subject in need thereof a therapeutically effective amount of a thymic peptide, is provided. Also provided is a method for inhibiting the pathological effects of activated monocytes in a subject in need thereof including treating the monocytes with an effective amount of the thymic peptide.

### FIELD OF THE INVENTION

This invention relates to a therapeutic method involving a thymus peptide.

### PRIOR ART

The following is a list of prior art, which is considered to be pertinent for describing the state of the art in the field of the invention. Acknowledgement of these references herein will be made by indicating the number from their list below within brackets.
(1) Akira, Shizuo, Kiyoshi Takeda & Tsuneyasu Kaisho, Nature Immunology 2, 675-680 (2001) Toll-like receptors: critical proteins linking innate and acquired immunity;
(2) Zhou and Yin, Chinese Medical Journal 117:1709-1715 (2004) Toll-like receptors: function and roles in asthma;
(3) Brint, Elizabeth K., Katherine A. Fitzgerald, Philip Smith, Anthony J. Coyle, Jose-Carlos Gutierrez-Ramos, Padraic G. Fallon, and Luke A. J. O'Neill, The Journal of Biological Chemistry Vol. 277, No. 51, (2002) pp. 49205-49211, Characterization of Signaling Pathways Activated by the Interleukin 1 (IL-1) Receptor Homologue T1/ST2;
(4) Kumar, S. et al, The Journal of Biological Chemistry Vol. 270, No. 46, (2002) pp. 27905-27913, ST2/T1 protein functionally binds to two secreted proteins from Balb/c 3T3 and human umbilical vein endothelial cells but does not bind interleukin 1;
(5) U.S. Patent Application No. 2005/0130136;
(6) WO 02/16646;
(7) WO 02/12487;
(8) U.S. Patent Application No. 2005/0130136;
(9) WO 06/071754;
(10) WO 06/033688;
(11) U.S. Patent Application No. 2005/0244336;
(12) WO 06/046239.

### BACKGROUND OF THE INVENTION

Recognition of pathogens is mediated by a set of germline-encoded receptors that are referred to as pattern-recognition receptors (PRRs). These receptors recognize conserved molecular patterns (pathogen-associated molecular patterns), which are shared by large groups of microorganisms. Toll-like receptors (TLRs) function as the PRRs in mammals and play an essential role in the recognition of microbial components. The TLRs may also recognize endogenous ligands induced during the inflammatory response. Similar cytoplasmic domains allow TLRs to use the same signaling molecules used by the interleukin 1 receptors (IL-1Rs). However, evidence is accumulating that the signaling pathways associated with each TLR are not identical and may, therefore, result in different biological responses (1). Due to their striking structural and functional similarities, the TLRs and the IL-1R are defined by the Toll/IL-1R (TIR) superfamily, having at least 30 members. TLRs have been indicated as having a role in asthma (2).

The T1/ST2 receptor (also referred to as “T1/ST2” and “ST2/T1”) is a member of the IL-1R superfamily, possessing three extracellular immunoglobulin domains and an intracellular TIR domain. The ligand for T1/ST2 is not known. T1/ST2 is expressed on Type 2 T helper (Th2) cells, and its role appears to be in the regulation of Th2 cell function. (3). Both soluble and membrane-bound T1/ST2 receptors are predominantly expressed in hematopoietic tissues in vivo. (4). T1/ST2 has been indicated as being involved in cardiovascular disease (5). Genetic variants of a putative T1/ST2 receptor binding protein (IL1RL1LG) gene have been disclosed (6). A polypeptide T1/ST2 receptor binding protein 10.23, an antagonist against the polypeptide and a polynucleotide encoding the polypeptide have also been disclosed (7).

Methods for diagnosing a cardiovascular condition, determining the clinical stage of a cardiovascular condition and treating a cardiovascular condition using the molecules Fit-1 (also known as T1/ST2), vacuolar ATPase, CD44, Lot-1, AA892598 and Mrg-1, and nucleic acids encoding them have been described (8).

Activated monocytes and macrophages contribute to mammalian disease states, and in particular atherosclerosis. Atherosclerosis is initiated when a fatty streak forms within a blood vessel wall. Formation of fatty streaks is believed to result from accumulation of lipoprotein particles in the intima layer of the blood vessel wall, the layer of the vessel wall underlying the luminal endothelial cell layer. Lipoprotein particles can associate with extracellular matrix components in the intima layer and can become inaccessible to plasma antioxidants, resulting in oxidative modification of the lipoprotein particles. Such oxidative modification may trigger a local inflammatory response resulting in adhesion of activated macrophages and T lymphocytes to the luminal endothelium followed by migration into the intima layer. The oxidized lipoprotein particles themselves can act as chemoattractants for cells of the immune system, such as macrophages and T cells, or can induce cells in the vascular wall to produce chemoattractants. The atherosclerotic lesion then forms a fibrous cap with a lipid-rich core filled with activated macrophages. Atherosclerotic lesions that are to unstable are characterized by local inflammation, and lesions that have ruptured and have caused fatal myocardial infarction are characterized by an infiltration of activated macrophages and T lymphocytes. (9-11)

Recently, a cDNA unique for the human thymus was identified. The peptide encoded by the cDNA has been named T101. The peptide is described in International Patent Application No. WO 2006/046239, filed Oct. 26, 2005, (corresponding to U.S. patent application Ser. No. 11/666,123), whose entire contents are incorporated by reference.

### SUMMARY OF THE INVENTION

One object of the present invention is to provide a method for treating diseases involving the T1/ST2 receptor.

Another object of the present invention is to provide a method for inhibiting pathological effects of activated monocytes.

Further objects of the invention will become apparent from the description below.

It has now been surprisingly discovered that the thymus peptide T101 may serve as a ligand of the T1/ST2 receptor. This discovery may be utilized for various therapeutic applications.

In a first aspect of the invention, there is provided a method for treating or preventing a disease, in a subject in need, involving a cell having a T1/ST2 receptor, comprising administering to the subject a therapeutically effective amount of an isolated polypeptide from the group consisting of:
(a) an isolated polypeptide comprising an amino acid sequence of SEQ. ID. NO: 1;
(b) an isolated polypeptide comprising an amino acid sequence of SEQ. ID. NO: 1, in which one or more amino acid residues is added, deleted or replaced, without significantly affecting the biological characteristics of the modified molecule as compared to the unmodified molecule;
(c) an isolated polypeptide comprising a partial contiguous sequence from SEQ. ID. NO: 1 that includes at least 8 amino acid residues, which contiguous sequence is included as a contiguous sequence in said SEQ. ID. NO: 1; and
(d) an isolated polypeptide comprising a contiguous sequence of 13 amino acid residues beginning from the N-terminal of SEQ. ID. NO: 1.

In the present specification, the term T1/ST2 receptor includes the soluble and membrane forms of the ST2 protein, as well as the T1/ST2 receptor.

The term “a disease involving a cell having a T1/ST2 receptor” is used herein to denote a disease whose etiology, severity and/or prognosis may be influenced by the T1/ST2 receptor, or by the binding of T101 to the receptor. Examples of such diseases include asthma diseases, acute and chronic cardiovascular diseases, idiopathic pulmonary fibrosis, Schistosomiasis and trauma, particularly surgery-induced trauma.

Examples of cardiovascular disease which may be treated in accordance with the invention include cardiac hypertrophy, myocardial infarction, stroke, atherosclerosis and heart failure.

Examples of asthma diseases which may be treated in accordance with the invention include intermittent asthma, persistent asthma, extrinsic (allergic) asthma, intrinsic asthma, exercise-induced asthma, occupational asthma, cough-variant asthma, chronic severe corticosteroid-dependent asthma, allergic bronchopulmonary aspergillosis and asthma triad.

The term “treating or preventing” in the context of the present invention refers to the administering of a therapeutic amount of the polypeptide or composition of the present invention which is effective to ameliorate undesired symptoms associated with a disease, to prevent the manifestation of such symptoms before they occur, to slow down the progression of the disease, slow down the deterioration or symptoms, to enhance the onset of remission period, slow down the irreversible damage caused in the progressive chronic stage of the disease, to delay the onset of said progressive stage, to lessen the severity or cure the disease, to improve survival rate or more rapid recovery, to prevent the disease form occurring, or a combination of two or more of the above. In addition, the term “treatment” in the context used herein also refers to prevention of the disease from occurring. The treatment (also preventative treatment) regimen and specific composition to be administered will depend on the type of disease to be treated and may be determined by various considerations known to those skilled in the art of medicine, e.g. the physicians.

The “therapeutically effective amount” for purposes herein is determined by such considerations as may be known in the art. The amount must be effective to achieve the desired therapeutic effect which depends on the type and mode of treatment. As is clear to the artisan, the amount should be effective to obtain the improvement of survival rate, to obtain a more rapid recovery, to obtain the improvement or elimination of symptoms or any other indicators as are selected as appropriate measures by those skilled in the art. Where, for example, the active ingredient is administered to treat a cardiovascular disease, an effective amount of the active ingredient may be an amount which reduces the symptoms of the disease or even cures the disease for a limited or extended period of time.

In a second aspect of the invention, there is provided a method for treating or preventing a disease, in a subject in need, involving a cell having a T1/ST2 receptor, comprising administering to the subject a therapeutically effective amount of an antagonist of T101.

The term “antagonist of T101” includes an antibody against T101 or a molecule which can bind to the T1/ST2 receptor in a manner that blocks the physiological effects of the receptor. An example of the physiological effects of the receptor is intracellular signal transduction in Th2 cells.

In a third aspect of the invention, there is provided a method for inhibiting the pathological effects of activated monocytes in a subject in need comprising administering to the subject a therapeutically effective amount of an isolated polypeptide from the group consisting of:
(a) an isolated polypeptide comprising an amino acid sequence of SEQ. ID. NO: 1;
(b) an isolated polypeptide comprising an amino acid sequence of SEQ. ID. NO: 1, in which one or more amino acid residues is added, deleted or replaced, without significantly affecting the biological characteristics of the modified molecule as compared to the unmodified molecule;
(c) an isolated polypeptide comprising a partial contiguous sequence from SEQ. ID. NO: 1 that includes at least 8 amino acid residues, which contiguous sequence is included as a contiguous sequence in said SEQ. ID. NO: 1; and
(d) an isolated polypeptide comprising a contiguous sequence of 13 amino acid residues beginning from the N-terminal of SEQ. ID. NO: 1.

In the present specification, the term “monocytes” may also include macrophages which differentiate from monocytes.

An “activated” monocyte is a monocyte which has undergone intracellular changes and has taken on new functions and/or properties. Examples of these functions and properties include (1) adherence to activated endothelial cells on the blood vessel wall and extravasation into the adjacent tissue, (2) an increase in intracellular expression of tumour necrosis factor-a (TNF-a) and interleukin-1β (IL-1β), and (3) having a larger diameter and an increased granularity. Various agents can activate a monocyte such as LPS, cytokines, fetal calf serum, and antigens or antibodies.

The term “pathological effects of activated monocytes” is used herein to denote pathological phenomena or disorders in the human body directly or indirectly caused by activated monocytes. Included within this term is one or more of rejection of transplanted cells or tissues, autoimmune disease, arthritis, an inflammatory bowel disease, an endocrinopathy, a neurodegenerative disease, a vascular disease, rejection of allogeneic cells, tissues or organs, rejection of xenogeneic cells, tissues or organs, graft versus host disease, systemic or discoid lupus erythematosus, sclerosing cholangitis, autoimmune hepatitis, rheumatoid arthritis, psoriasis, psoriatic arthritis, ulcerative colitis, Crohn's disease, type 1 diabetes, Graves disease, multiple sclerosis, autistic spectrum disorder, Alzheimer's disease, amyotrophic lateral sclerosis (ALS), Parkinson's disease, Huntingdon's Disease, Guillain-Barre syndrome, myasthenia gravis, chronic idiopathic demyelinating disease (CID), autoimmune hearing loss, systemic vasculitis, or atherosclerosis. In a preferred embodiment, the term refers to the development of atherosclerotic plaques in blood vessels. In another embodiment, one or more of the above pathological phenomena or disorders may be excluded from the term “pathological effects of activated monocytes”.

Without wishing to limit the scope of the invention, it is postulated that one manner in which T101 inhibits the pathological effects of activated monocytes is by inducing apoptosis in the activated monocytes.

A fourth aspect of the invention relates to a method for protecting and preventing damage to the liver. Damaged liver secretes several enzymes to the blood. Among them are aspartate aminotransferase (AST), alkaline phosphatase (ALP) and alanine aminotransferase (ALT). It has now been found that treating mice with T101 can cause a decrease in secretion of these enzymes to the blood. Thus, T101 can also help the liver to recover from various pathological insults including: damage caused by drugs and chemicals; cirrhosis; liver inflammation; fatty liver (non alcoholic steatohepatitis (NASH); hepatitis A; hepatitis B; hepatitis C; primary biliary cirrhosis; primary sclerosing cholangitis; autoimmune hepatitis; Wilson's Disease; and alcohol related liver disease. In one embodiment, the pathological insult is not liver cancer.

A fifth aspect of the invention relates to a method for decreasing blood levels of cholesterol. It has been found that long term treatment of mice with T101 can decrease cholesterol blood levels. This aspect of the invention can be very important in terms of treating diseases associated with high blood levels of cholesterol, such as atherosclerosis. Thus, T101 can serve as a drug to regulate cholesterol blood levels and treat hypercholesterolaemia, and indirectly affect diseases influenced by cholesterol levels, including cardiovascular diseases such as atherosclerosis.

In one embodiment of the invention, the polypeptide consists of the following sequence (SEQ ID. NO: 1):

(SEQ. ID. NO: 1)
LHLWLSGEPVQSSGTKDMRSKSDSKRVSDKQLISKAVWWT
FFLPSTLWERK

This polypeptide will be referred to herein as the “T101 peptide” or “T101”.

The term “peptide” is used herein to denote a peptide, polypeptide or protein. The peptide may be obtained synthetically, through genetic engineering methods, expression in a host cell, or through any other suitable means.

A nucleic acid molecule comprising a sequence encoding for the T101 peptide includes the following sequence (SEQ. ID. NO: 2):

(SEQ. ID. NO: 2)

CATCTCTGGCTTAGTGGGGAGCCAGTCCAGAGCTCTGGAACAAA
GGACATGAGATCCAAATCCGATTCCAAGCGAGTGAGTGACAAGCAGCT
AATTTCCAAAGCTGTGTGGTGGACATTTTTTCTTCCTTCAACCCTCTG
GGAGAGAAAATGA

The T101 peptide is included in a larger polypeptide encoded by a cDNA which is 84 amino acids long and includes a signal peptide of 33 amino acids on its N-terminal end. The cDNA sequence (SEQ. ID. NO: 3) and amino acid sequence (SEQ. ID. NO: 4) of this longer peptide are as follows:

(SEQ. ID. NO: 3)
ATGATGGCACTCAGAAGCCAGGGGCTCATGTTACCCCAGA
GCTGCCCACAACTGGCTTTCCTCACCCTAAGTGCCTTGGC
AGCAGTGTCTTTTTCAGCTCTGCATCTCTGGCTTAGTGGG
GAGCCAGTCCAGAGCTCTGGAACAAAGGACATGAGATCCA
AATCCGATTCCAAGCGAGTGAGTGACAAGCAGCTAATTTC
CAAAGCTGTGTGGTGGACATTTTTTCTTCCTTCAACCCTC
TGGGAGAGAAAATGA
(SEQ. ID. NO: 4)
MMALRSQGLMLPQSCPQLAFLTLSALAAVSFSALHLWLSG
EPVQSSGTKDMRSKSDSKRVSDKQLTSKAVWWTFFLPSTL
WERK

The polypeptide of SEQ. ID. NO: 3 will be referred to herein as the “full T101 peptide”. The full T101 peptide may also be used in the method of the invention.

The term “active ingredient” may be used at times in the specification to denote the active substance used in the method of the invention, such as T101 or a derivative thereof.

In a second embodiment of the invention, the polypeptide consists of an amino acid sequence of SEQ. ID. NO: 1 or SEQ. ID. NO: 4, in which one or more amino acid residues is added, deleted or replaced, without significantly affecting the biological characteristics of the modified molecule as compared to the unmodified molecule.

The term “biological characteristics”, with respect to a peptide molecule, refers to the peptide's ability to exert at least one of the in vitro or in vivo effects that may be exerted by the T101 peptide or the full T101 peptide, including but not limited to the biological activities reported below in the Examples. The term “biological characteristics”, with respect to a nucleic acid molecule, refers to the property of encoding a peptide having similar biological characteristics to that of the T101 peptide or the full T101 peptide, including, in particular: (i) a nucleic acid molecule that has a different sequence to that of SEQ. ID. NO: 2 or SEQ. ID. NO: 3, but, owing to the redundancy of the genetic code, encodes the T101 peptide or the full T101 peptide, respectively; and (ii) a nucleic acid molecule that encodes an amino acid molecule with a different sequence than that of the T101 peptide or the full T101 peptide but that has similar biological characteristics to that of the T101 peptide or the full T101 peptide, respectively.

The term “without significantly affecting the biological characteristics of the modified molecule as compared to the unmodified molecule” means to denote that the modified molecule retains a biological activity qualitatively similar to that of the unmodified molecule. With respect to a modified peptide, this means that it retains one or more of the biological characteristics of a peptide of SEQ. ID. NO: 1 or SEQ. ID. NO: 4, including, among others, its therapeutic utilities, as specified in this specification, as well as its in vitro and in vivo activities reported in the Examples below. In order to determine whether a peptide retains a biological activity qualitatively similar to that of the unmodified molecule, one or more assays can be carried out, such as for example an in vitro, in vivo or a clinical experiment in which a modified peptide is compared to the corresponding unmodified one (namely that of the T101 peptide or the full T101 peptide) that is assayed in parallel; or an experiment in which the modified peptide is assayed to examine whether it has a biological effect similar to that of the unmodified peptide as known from separately conducted experiment. Such an experiment may be carried out, for example, in manner described in the Examples below. With respect to a modified nucleic acid molecule, the term “without significantly affecting the biological characteristics of the modified molecule as compared to the unmodified molecule” denotes the property of encoding a modified peptide of any of the above characteristics.

In a third embodiment of the invention, the polypeptide consists of a peptide comprising a partial contiguous sequence from the T101 peptide including at least 8 amino acid residues, which contiguous sequence is included as a contiguous sequence in said T101 peptide. Such a peptide will be referred to herein as a “Partial T101 peptide”.

In a fourth embodiment of the invention, the polypeptide consists of a partial T101 peptide that comprises a contiguous sequence of 13 amino acid residues beginning from the N-terminal end of the T101 peptide (amino acid nos. 39 to 51), as follows:

| | |
|---|---|
| WTFFLPSTLWERK, | (SEQ. ID. NO: 5) |

and will be referred to herein as the “13aa T101 peptide”.

A partial T101 peptide may be a peptide that includes a contiguous sequence of at least 8, 12, 15, 20, 25, 30, 35, 40 or at least 45 amino acid residues that has a degree of identity to a corresponding sequence of at least 8, 12, 15, 20, 25, 30, 35, 40 or at least 45 amino acid residues included in the T101 peptide, the degree of identity being at least 70%, preferably at least 80%, more preferably at least 90% and particularly at least 95%.

A protein or polypeptide comprising an amino acid sequence of the full T101 peptide, T101 peptide, modified peptide or a partial T101 peptide (such protein or polypeptide will be referred to herein as “T101 comprising protein”) may also be used in the method of the invention. The T101 comprising protein may, for example, be a fusion protein that comprises the full T101 peptide, the T101 peptide, a modified peptide or a partial T101 peptide; it may be a conjugate of a protein or another peptide or polypeptide with the full T101 peptide, T101 peptide, modified peptide or partial T101 peptide; etc.

Additional partial T101 peptides which may be used in the method of the invention are as follows:

| | |
|---|---|
| SGEPVQSSGTKDMRSKSDSKRVS | (SEQ. ID. NO: 6) |
| DKQLISKAVWWTFFLPSTLWERK | (SEQ. ID. NO: 7) |
| PSTLWERK | (SEQ. ID. NO: 8) |
| AVWWTFFLPSTLW | (SEQ. ID. NO: 9) |
| KREWLTSPLFFTWWVA | (SEQ. ID. NO: 10) |
| WTFFL | (SEQ. ID. NO: 11) |

SEQ. ID. NO: 6 consists of amino acids 6 to 28 of the T101 peptide; SEQ. ID. NO: 7 consists of amino acids 29 to 51 of the T101 peptide; SEQ. ID. NO: 8 consists of amino acids 44 to 51 of the T101 peptide; SEQ. ID. NO: 9 consists of amino acids 36 to 48 of the T101 peptide; SEQ. ID. NO: 10 consists of amino acids 36 to 51 of the T101 peptide in the reverse order; and SEQ. ID. NO: 11 consists of amino acids 39 to 43 of the T101 peptide.

Further examples of partial T101 peptides are modified peptides derived from any of the peptides defined above, e.g., modified peptides in which one or more amino acids are replaced by another amino acid by conservative substitution. As used herein, “conservative substitution” refers to the substitution of an amino acid in one class by an amino acid of the same class, where a class is defined by common physicochemical amino acid side chain properties and high substitution frequencies in homologous proteins found in nature. Six general classes of amino acid side chains have been categorized and include: Class I (Cys); Class II (Ser, Thr, Pro, Ala, Gly); Class III (Asn, Asp, Gln, Glu); Class W (His, Arg, Lys); Class V (Ile, Leu, Val, Met); and Class VI (Phe, Tyr, Trp). For example, substitution of an Asp for another class III residue such as Asn, Gin, or Glu is a conservative substitution.

In one embodiment, only one substitution is made in the amino acid sequence. In another embodiment, two substitutions are made. In a further embodiment, three substitutions are made. The maximum number of substitutions should not exceed that number of amino acids which leaves at least 70%, desirably at least 80%, preferably at least 90%, most preferably at least 95% of the amino acids in the unsubstituted sequence. By one preferred embodiment, the substitutions which include up to 3, at times up to 6 amino acid residues substituted by others, are conservative substitutions.

In a further embodiment, one or more amino acids may be replaced by D-amino acids, preferably the corresponding D-amino acids.

In a still further embodiment, sequences of the reverse order of the above sequences may also be used in the invention.

Thus, full T101 peptides of SEQ ID NO: 4 or preferably T101 peptides of SEQ ID NO: 1 or partial T101 sequences thereof, modified by one or more conservative substitutions may also be used in the method of the invention.

These peptides include at least 10, or 15, or 20, or 25, or 30, or 35, or 40 amino acid residues, or the entire sequence of the T101 peptide having the sequence: AA₁, -AA₂- . . . -AA₅₁, wherein:

AA₁ is selected from leucine, isoleucine, valine and methionine;

AA₂ is selected from lysine, arginine and histidine;

AA₃ is selected from leucine, isoleucine, valine and methionine;

AA₄ is selected from tryptophan, phenylalanine and tyrosine;

AA₅ is selected from leucine, isoleucine, valine and methionine;

AA₆ is selected from serine, threonine, alanine, glycine and proline;

AA₇ is selected from serine, threonine, alanine, glycine and proline;

AA₈ is selected from glutamine, glutamic acid, aspartic acid and asparagine;

AA₉ is selected from serine, threonine, alanine, glycine and proline;

AA₁₀ is selected from leucine, isoleucine, valine and methionine;

AA₁₁ is selected from glutamine, glutamic acid, aspartic acid and asparagine;

AA₁₂ is selected from serine, threonine, alanine, glycine and proline;

AA₁₃ is selected from serine, threonine, alanine, glycine and proline;

AA₁₄ is selected from serine, threonine, alanine, glycine and proline;

AA₁₅ is selected from serine, threonine, alanine, glycine and proline;

AA₁₆ is selected from lysine, arginine and histidine;

AA₁₇ is selected from glutamine, glutamic acid, aspartic acid and asparagine;

AA₁₈ is selected from leucine, isoleucine, valine and methionine;

AA₁₉ is selected from lysine, arginine and histidine;

AA₂₀ is selected from serine, threonine, alanine, glycine and proline;

AA₂₁ is selected from lysine, arginine and histidine;

AA₂₂ is selected from serine, threonine, alanine, glycine and proline;

AA₂₃ is selected from glutamine, glutamic acid, aspartic acid and asparagine;

AA₂₄ is selected from serine, threonine, alanine, glycine and proline;

AA₂₅ is selected from lysine, arginine and histidine;

AA₂₆ is selected from lysine, arginine and histidine;

AA₂₇ is selected from leucine, isoleucine, valine and methionine;

AA₂₈ is selected from serine, threonine, alanine, glycine and proline;

AA₂₉ is selected from glutamine, glutamic acid, aspartic acid and asparagine;

AA₃₀ is selected from lysine, arginine and histidine;

AA₃₁ is selected from glutamine, glutamic acid, aspartic acid and asparagine;

AA₃₂ is selected from leucine, isoleucine, valine and methionine;

AA₃₃ is selected from leucine, isoleucine, valine and methionine;

AA₃₄ is selected from serine, threonine, alanine, glycine and praline;

AA₃₅ is selected from lysine, arginine and histidine;

AA₃₆ is selected from serine, threonine, alanine, glycine and proline;

AA₃₇ is selected from leucine, isoleucine, valine and methionine;

AA₃₈ is selected from tryptophan, phenylalanine and tyrosine;

AA₃₉ is selected from tryptophan, phenylalanine and tyrosine;

AA₄₀ is selected from serine, threonine, alanine, glycine and proline;

AA₄₁ is selected from tryptophan, phenylalanine and tyrosine;

AA₄₂ is selected from tryptophan, phenylalanine and tyrosine;

AA₄₃ is selected from leucine, isoleucine, valine and methionine;

AA₄₄ is selected from serine, threonine, alanine, glycine and proline;

AA₄₅ is selected from serine, threonine, alanine, glycine and proline;

AA₄₆ is selected from serine, threonine, alanine, glycine and proline;

AA₄₇ is selected from leucine, isoleucine, valine and methionine;

AA₄₈ is selected from tryptophan, phenylalanine and tyrosine;

AA₄₉ is selected from glutamine, glutamic acid, aspartic acid and asparagine;

AA₅₀ is selected from lysine, arginine and histidine; and

AA₅₁ is selected from lysine, arginine and histidine.

Included are also modified peptides based on the full T101 peptide, T101 peptide or partial T101 peptide, including the following subsequences (amino acid numbering based on the T101 peptide):

AA₃₈-AA₃₉-AA₄₀-AA₄₁-AA₄₂; wherein AA₃₈ and AA₃₉ are Class VI amino acids, preferably tryptophan; AA₄₀ is a Class II amino acid, preferably threonine; and AA₄₁ and AA₄₂ are Class VI amino acids, preferably phenylalanine.

AA₃₈-AA₃₉-AA₄₀-AA₄₁-AA₄₂-AA₄₃; wherein AA₃₈ and AA₃₉ are Class VI amino acids, preferably tryptophan; AA₄₀ is a Class II amino acid, preferably threonine; AA₄₁ and AA₄₂ are Class VI amino acids, preferably phenylalanine; and AA₄₃ is a Class V amino acid, preferably leucine.

Ala-Val-AA₃₈-AA₃₉-AA₄₀-AA₄₁-AA₄₂; wherein AA₃₈ and AA₃₉ are Class VI amino acids, preferably tryptophan; AA₄₀ is a Class II amino acid, preferably threonine; and AA₄₁ and AA₄₂ are Class VI amino acids, preferably phenylalanine.

Ala-Val-AA₃₈-AA₃₉-AA₄₀-AA₄₁-AA₄₂-AA₄₃; wherein AA₃₈ and AA₃₉ are Class VI amino acids, preferably tryptophan; AA₄₀ is a Class II amino acid, preferably threonine; AA₄₁ and AA₄₂ are Class VI amino acids, preferably phenylalanine; and AA₄₃ is a Class V amino acid, preferably leucine.

The peptides and polypeptides used in the method of the invention may be manufactured by any conventional process such as chemical synthesis and recombinant technology.

A therapeutically effective amount of T101 is typically administered in a single daily dose, although at times a daily dose may be divided into several doses administered throughout the day or at times several daily doses may be combined into a single dose to be given to the patient once every several days, particularly if administered in a sustained release formulation.

The active ingredient may be administered as a non-active substance (e.g. pro-drug) and be made active only upon further modification/s by a natural process at a specific site in the subject. In any case, the derivative will be such that the therapeutic functionality of the pharmaceutical composition of the invention is preserved. Such pro-drugs are also encompassed by the term “active ingredient” as used herein.

The method of the invention may also include the administration of drugs in addition to the isolated polypeptide. For example, cardiovascular drugs may be administered before, together with or subsequently to the administration of the isolated polypeptide.

A further embodiment of the invention relates to a pharmaceutical composition comprising an effective amount of an isolated polypeptide from the group consisting of:
(a) an isolated polypeptide comprising an amino acid sequence of SEQ. ID. NO: 1;
(b) an isolated polypeptide comprising an amino acid sequence of SEQ. ID. NO: 1, in which one or more amino acid residues is added, deleted or replaced, without significantly affecting the biological characteristics of the modified molecule as compared to the unmodified molecule;
(c) an isolated polypeptide comprising a partial contiguous sequence from SEQ. ID. NO: 1 that includes at least 8 amino acid residues, which contiguous sequence is included as a contiguous sequence in said SEQ. ID. NO: 1; and
(d) an isolated polypeptide comprising a contiguous sequence of 13 amino acid residues beginning from the N-terminal of SEQ. ID. NO: 1;

for use in a method for treating or preventing a disease involving a cell having a T1/ST2 receptor in a subject in need.

The administration of the polypeptide to a patient may be together with a pharmaceutically acceptable carrier.

By the term “pharmaceutically acceptable carrier” it is meant any one of inert, non-toxic materials, which do not react with the active ingredient. The carrier is selected at times based on the desired form of the formulation. The carrier may also at times have the effect of the improving the delivery or penetration of the active ingredient to the target tissue, for improving the stability of the drug, for slowing clearance rates, for imparting slow release properties, for reducing undesired side effects etc. The carrier may also be a substance that stabilizes the formulation (e.g. a preservative), for providing the formulation with an edible flavor, etc. The carriers may be any of those conventionally used and is limited only by chemical-physical considerations, such as solubility and lack of reactivity with the polypeptide, and by the route of administration. The carrier may include additives, colorants, diluents, buffering agents, disintegrating agents, moistening agents, preservatives, flavoring agents, and pharmacologically compatible carriers. In addition, the carrier may be an adjuvant, which, by definition are substances affecting the action of the active ingredient in a predictable way. Typical examples of carriers include (a) liquid solutions, where an effective amount of the active substance is dissolved in diluents, such as water, saline, natural juices, alcohols, syrups, etc.; (b) capsules (e.g. the ordinary hard- or soft-shelled gelatin type containing, for example, surfactants, lubricants, and inert fillers), tablets, lozenges (wherein the active substance is in a flavor, such as sucrose and acacia or tragacanth or the active substance is in an inert base, such as gelatin and glycerin), and troches, each containing a predetermined amount of active agent as solids or granules; (c) powders; (d) suspensions in an appropriate liquid; (e) suitable emulsions; (f) liposome formulation; and others.

When administering the compositions of the present invention parenterally, it will generally be formulated in a unit dosage injectable form (solution, suspension, emulsion). The pharmaceutical formulation suitable for injection includes sterile aqueous solutions or dispersions and sterile powders for reconstitution into sterile injectable solutions or dispersions. The carrier employed can be a solvent or dispersing medium containing, for example, water, ethanol, polyol (for example, glycerol, propylene glycol, lipid polyethylene glycol and the like), suitable mixtures thereof and vegetable oils.

In any case, the pharmaceutical compositions of the invention are administered and dosed in accordance with good medical practice, taking into account the clinical condition of the individual patient, the site and method of administration, scheduling of administration, patient's age, sex, body weight and other factors known to medical practitioners.

The composition of the invention may be administered in various ways. It can be administered orally, subcutaneously or parenterally including intravenous, intraarterial, intramuscular, intraperitoneally or by intranasal administration, as well as by intrathecal and infusion techniques known to the man versed in the art.

As known, a treatment course in humans is usually longer than in animals, e.g. mice, as exemplified herein. The treatment has a length proportional to the length of the disease process and active agent effectiveness. The therapeutic regimen may involve single doses or multiple doses over a period of several days or more. The treatment generally has a length contingent with the course of the disease process, active agent effectiveness and the patient species being treated.

Non-aqueous vehicles such as cottonseed oil, sesame oil, olive oil, soybean oil, corn oil, sunflower oil, or peanut oil and ester, such as isopropyl myristate, may also at times be used as solvent systems for the active ingredient.

Additionally, various additives which enhance the stability, sterility and isotonicity of the compositions, including antimicrobial preservatives, antioxidants, chelating agents and buffers can be added. Prevention of the action of microorganisms can be ensured by various antibacterial and antifungal agents, for example, parabens, chlorobutanol, phenol, sorbic acid and the like.

For the purpose of oral administration, the active ingredient may be formulated in the form of tablets, suspensions, solutions, emulsions, capsules, powders, syrups and the like, are usable and may be obtained by techniques well known to the pharmacists.

A still further embodiment of the invention relates to a use of an effective amount of an isolated polypeptide from the group consisting of:
(a) an isolated polypeptide comprising an amino acid sequence of SEQ. ID. NO: 1;
(b) an isolated polypeptide comprising an amino acid sequence of SEQ. ID. NO: 1, in which one or more amino acid residues is added, deleted or replaced, without significantly affecting the biological characteristics of the modified molecule as compared to the unmodified molecule;
(c) an isolated polypeptide comprising a partial contiguous sequence from SEQ. ID. NO: 1 that includes at least 8 amino acid residues, which contiguous sequence is included as a contiguous sequence in said SEQ. ID. NO: 1; and
(d) an isolated polypeptide comprising a contiguous sequence of 13 amino acid residues beginning from the N-terminal of SEQ. ID. NO: 1;

in the preparation of a pharmaceutical composition for use in a method for treating or preventing a disease involving a cell having a T1/ST2 receptor in a subject in need.

In a further aspect, the invention relates to a T101 peptide obtained from mouse, having the following sequence:

SEQ ID NO: 12 (with signal peptide)-
MDLSIRLSLACWELNQVSGAWGMSLKSHFKFMSDKQLISKAVQRIFFSP
STLWGEK
(56 amino acids)
SEQ ID NO: 13 (without signal peptide)-
MSLKSHFKFMSDKQLISKAVQRIFFSPSTLWGEK
(34 amino acids)

Analogues of the above sequences included in the invention include the following sequences:

SEQ ID NO: 14 (with signal peptide)-
MDLSIRLSLACWELNQVSGAWGMSLKSHFKFMSDKQLISKAVX₁X₂X₃
FFSPSTLWX₄X₅K
SEQ ID NO: 15 (without signal peptide)-
MSLKSHFKFMSDKQLISKAVX₁X₂X₃FFSPSTLWX₄X₅K

where Xₙ is defined as follows;

X₁—Q,W

X₂—R,W

X₃—I,T

X₄—G,E

X₅—E,R

Also included in the invention is the cDNA sequence of the mouse T101 peptide:

SEQ ID NO: 16 (with signal peptide)-
ATGGACCTT TCCATCCGT CTGTCTCTT GCTTGCTGG GAGCTGAAC
CAGGTCTCT GGAGCATGG GGCATGAGC TTAAAATCC CATTTCAAG
TTCATGAGT GACAAGCAG CTAATTTCC AAAGCTGTG CAGCGGATA
TTTTTTTCT CCTTCAACC CTCTGGGGG GAAAAA TGA
SEQ ID NO: 17 (without signal peptide)-
ATGAGC TTAAAATCC CATTTCAAG TTCATGAGT GACAAGCAG
CTAATTTCC AAAGCTGTG CAGCGGATA TTTTTTTCT CCTTCAACC
CTCTGGGGG GAAAAA TGA

It should be pointed out that a similar cDNA sequence published as part of the mouse genome has a superfluous C nucleotide inserted in the sequence, causing a frame-shift error.

Also included in the invention are analogs and homologues of the above protein and DNA sequences in which one or more amino acid or nucleotide residues is added, deleted or replaced, without significantly affecting the biological characteristics of the modified molecule as compared to the unmodified molecule.

A comparison between human and mouse T101 peptide sequences is as follows:

| | |
|---|---|
| Human: | MMALRSQGLMLPQSCPQLAFLTLSALAAVSFSALHLWLSGEPVQSSGTLDMRSKSDSKRV |
| Mouse: | -MDLS---IRLSLACWELN------------------------QVSGAWGMSLKSHFKFM |
| | * * : *. :* :* * **: .* **. * : |
| Human: | SDKQLISKAVWWTFFLPSTLWERK |
| Mouse: | SDKQLISKAVQRIFFSPSTLWGEK |
| | ********** ** ***** .* |

* = exact correspondence
: = same amino acid class
. = similar amino acid class

The present invention is defined by the claims, the contents of which are to be read as included within the disclosure of the specification, and will now be described by way of example with reference to the accompanying Figures. It is to be understood, that the terminology which has been used is intended to be in the nature of words of description rather than limitation.

While the foregoing description describes in detail only a few specific embodiments of the invention, it will be understood by those skilled in the art that the invention is not limited thereto and that other variations in form and details may be possible without departing from the scope and spirit of the invention herein disclosed.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a Western blot of lysed U937 cells run on SDS-PAGE and treated with anti-caspase 3 antibody;

FIGS. 2 & 3 show Western blots of lysed U937 cells run on SDS-PAGE and treated with antibody against the 85 kDa cleavage product of anti-poly(ADP-ribose) polymerase (PARP);

FIGS. 4 & 5 show Western blots of lysed murine spleenocytes run on SDS-PAGE and treated with either anti-caspase 3 antibody (FIG. 4) or anti-caspase 6 antibody (FIG. 5);

FIG. 6 shows a Western blot of T101 or anti-T1/ST2 receptor antibody run on SDS-PAGE and treated with anti F_{C} antibody;

FIG. 7 is a bar diagram showing the level of cholesterol in the blood of mice treated with T101 or saline;

FIG. 8 is a bar diagram showing the level of various liver enzymes in the blood of the mice of FIG. 7;

FIG. 9 shows a Western blot of samples from lysed U937 cells run on SDS-PAGE and treated with antibody against FLICE-inhibitory protein (FLIP) and caspase 8. The samples were obtained from T1/ST2 immuno-precipitated from U937 cells at different time points after incubation with T101. The antibodies used to detect caspase 8 were against the active cleaved species;

FIG. 10 shows a Western blot of lysed U937 cells run on SDS-PAGE and treated with antibody against phosphorylated Jun N-terminal kinase (JNK), phosphorylated mitogen-activated protein kinase (MAPK) p38, and IκB subunit. The U937 cells were incubated for 30 min. with (+) or without (−) T101;

FIG. 11 shows a Western blot of lysed U937 cells run on SDS-PAGE and treated with antibody against Bcl-2 and full length caspase 9; and

FIG. 12 shows a Western blot of the proteins vascular endothelial growth factor (VEGFA), VEGFA receptor 1 (VEGFR1), hypoxanthine-guanine phosphoribosyl transferase (HPRT) and IL-10 in the absence or presence of T101. HPRT was included as a negative control.

### DETAILED DESCRIPTION OF EXEMPLARY EMBODIMENTS

### Example 1

The purpose of this example was to show that the T101 peptide can cause apoptosis in activated monocytes.

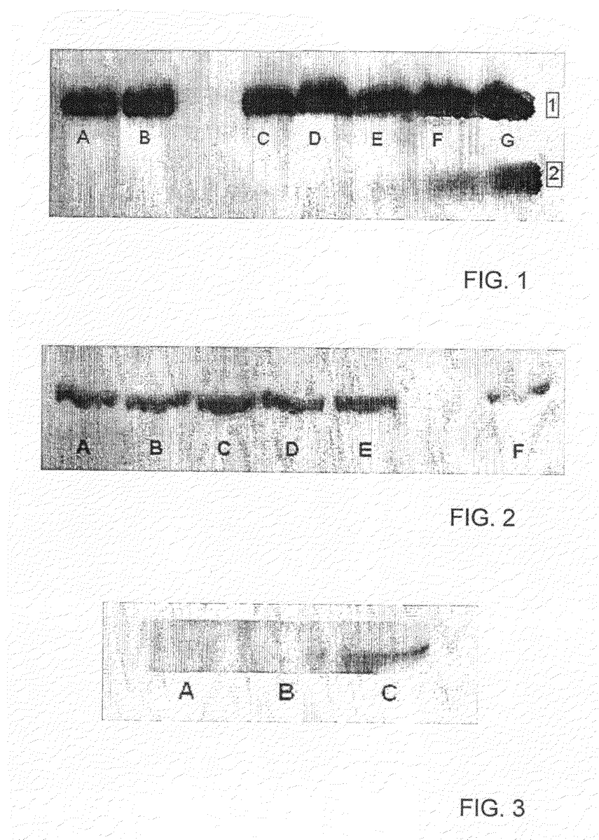

10⁶ U937 cells, a human leukemic monoblast cell line, were incubated for 18 hr at 37° C. in RPMI buffer+10% fetal calf serum (FCS) with different concentrations of T101. The FCS causes activation of the U937 cells. The cells were then lysed in RIPA buffer in the presence of protease inhibitors, and run on SDS-PAGE. A Western blot of the gel was performed with anti-caspase 3 antibody. The results are presented in FIG. 1.

The lanes of the gel were loaded with lysed cells which had been incubated with the following concentrations of T101: A,B—0 pg/ml (control); C—10 pg/ml; D—100 pg/ml; E—1 ng/ml; F—10 ng/ml; G—100 ng/ml. The upper row in the gel (marked [1]) shows the location of the proenzyme caspase, while the lower row (marked [2]) shows the location of activated caspase 3 as a result of proteolytic cleavage of the proenzyme.

It can be seen that increasing amounts of T101 result in increased activated caspase 3 in the U937 cells, leading to apoptosis in the cells. Thus, T101 can be expected to cause apoptosis in activated monocytes, thereby reducing the possibility of development of a disease associated with activated monocytes, such as atherosclerosis.

### Example 2

The purpose of this example was to show the apoptosis-inducing effect of T101 using a different marker.

U937 cells were treated as in FIG. 1 and loaded on SDS-PAGE, except that the gel was treated with antibody against the 85 kDa cleavage product of PARP, which is produced by the cleavage of PARP by caspase 3, and is active in apoptosis. The results are presented in FIG. 2.

The lanes of the gel were loaded with lysed cells which had been incubated with the following concentrations of T101: A —100 ng/ml; B —10 ng/ml; C —1 ng/ml; D —100 pg/ml; E —10 pg/ml; F —0 pg/ml (control). The bands show the location of the 85 kDa cleavage product of PARP. It can be seen that even 10 pg/ml of T101 significantly increased the amount of the 85 kDa cleavage product of PARP, which can lead to apoptosis of the cells. These results support the results of the previous example.

### Example 3

This example is similar to Example 2 except that it compares the effect of T101 on activated as compared to nonactivated monocytes.

U937 cells were treated as in FIG. 2 except that some of the cells were not treated with FCS, i.e. not activated. The lysed cells were loaded on SDS-PAGE, which was treated with antibody against the 85 kDa cleavage product of PARP. The results are presented in FIG. 3.

The lanes of the gel were loaded with lysed cells, as follows: A—activated cells+0 ng/ml T101 (control) [the result with nonactivated cells was similar]; B—nonactivated cells+100 ng/ml T101; C—activated cells+100 ng/ml T101.

It can be seen that T101 induced the cleavage of PARP only in the activated monocytes. Thus, it can be seen that the apoptosis inducing effect of T101 is specific for activated monocytes, which are a risk factor for diseases such as atherosclerosis.

### Example 4

The previous examples showed the effect of T101 on activated cancerous monocytes in vitro. In this and the following examples, the effect of T101 on normal monocytes in vivo was investigated.

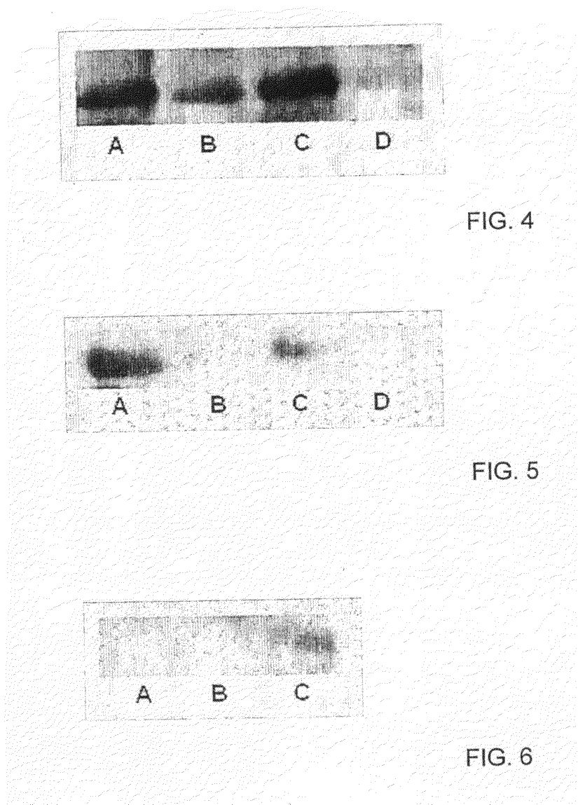

Four groups of 3 mice each (male Balb/c, 6 wks old, approx. 20 gr.) were injected ip with the following: A—saline (control); B—50 μg LPS (induces activation of monocytes); C —50 μg LPS+1 μg T101; D—1 μg T101. After 24 hours the mice were sacrificed and the spleens were removed. The spleens of the mice of each group were combined and lysed in RIPA buffer in the presence of protease inhibitors, and 30×10⁶ lysed spleenocytes from each group were run on SDS-PAGE. A Western blot of the gel was performed with antibody against activated caspase 3. The results are presented in FIG. 4.

It may be seen that the mice who received both LPS and T101 (C) showed an increased amount of activated caspase 3 as compared to the control, i.e. an increased amount of apoptosis due to caspase 3 in activated spleenocytes, while the mice who received only T101 (without LPS) (D) showed an inhibition of activated caspase 3, i.e. a lack of apoptosis in nonactivated spleenocytes. These results indicate that T101 can be used to induce apoptosis by caspase 3 in activated spleenocytes, thus preventing diseases induced by such cells, while not affecting nonactivated cells.

### Example 5

This example shows the results of a Western blot carried out with the cells of Example 4, using an anti-activated caspase 6 antibody instead of an anti-activated caspase 3 antibody. The results are shown in FIG. 5.

The results are similar to those of Example 4 and show that T101 induces caspase 6 in activated spleenocytes (C) while inhibiting caspase 6 in nonactivated spleenocytes (D).

### Example 6

In this example, the binding of T101 to the T1/ST2 receptor was investigated.

An antibody to the T1/ST2 receptor (F_{C}-T1/ST2), and T101 labeled with biotin (T101-bio) were prepared. Streptavidin beads were incubated for 1 hr. at 4° C. with each of the following: A —T101-bio alone; B—Fc-T1/ST2 alone; C—F_{C}-T1/ST2 pre-incubated with T101-bio for 45 min. at 4° C. After incubation, the beads were spun down and washed 3 times with PBS+0.1% Tween 20. The beads were then loaded on SDS-PAGE and a Western blot was performed using antibody against T1/ST2. The results are presented in FIG. 6.

Since only T101-bio binds to the Streptavidin beads and not F_{C}-T1/S′T2, the band which can be seen in C indicates that the T101-bio was bound by the F_{C}-T1/ST2 during the pre-incubation thus enabling the F_{C}-T1/ST2 to be bound by the Streptavidin beads. This indicates that T101 binds to the T1/ST2 receptor.

### Example 7

The effect of T101 on blood cholesterol levels was investigated in this example.

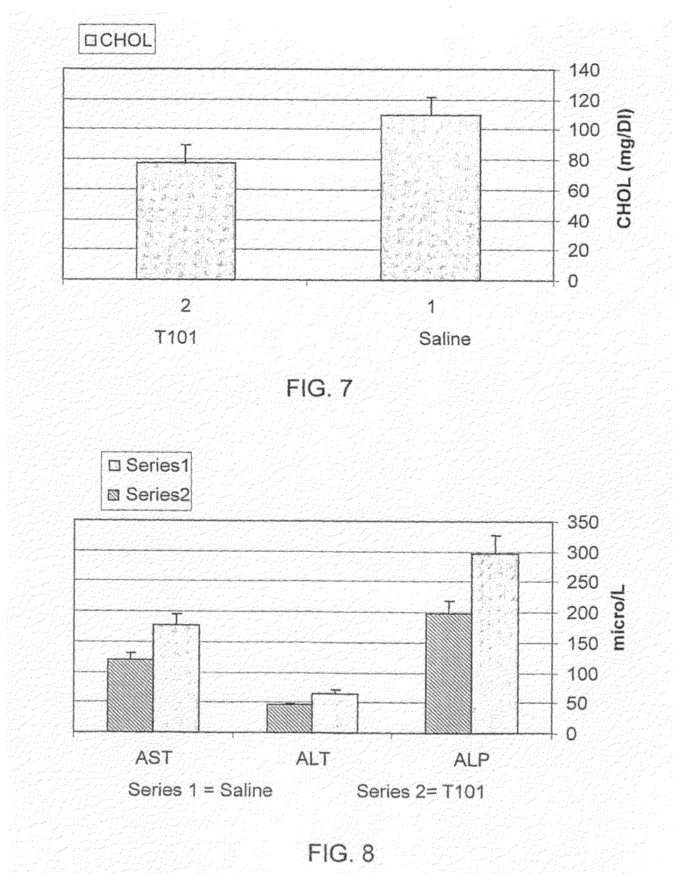

Two groups of 7 weeks old female Balb/C mice were injected twice a day for 3 weeks with either T101 (50 μg/Kg) (8 mice), or with saline (control) (7 mice). After 3 weeks, blood was collected from the mice and pooled, and the cholesterol level (mg/DL) was measured. The results are presented in FIG. 7.

It can be seen from FIG. 7 that prolonged treatment of mice with T101 resulted in a significant decrease in cholesterol levels in the blood. Thus, T101 can be used to reduce blood cholesterol levels thereby influencing various pathogenic physiological processes affected by cholesterol levels.

### Example 8

In this example, the effect of T101 on blood liver enzyme levels was investigated.

Liver enzymes levels (μg/L) were measured in the blood of the mice of Example 7. The enzymes measured were aspartate aminotransferase (AST), alkaline phosphatase (ALP) and alanine aminotransferase (ALT). The results are presented in FIG. 8.

It can be seen from FIG. 8 that prolonged treatment of mice with T101 resulted in a significant decrease in liver enzyme levels in the blood, apparently as a result of restoring the liver to health. Thus, T101 can be used to reduce damage to the liver.

### Example 9

In this example, the time course of the effect of T101 on T1/ST2 receptor-related apoptosis was investigated by following the degradation of FLIP and activation of caspase 8. U937 cells were incubated with T101, and at different time points T1/ST2 was immuno-precipitated from the cells and analyzed by Western blot for the presence of caspase 8 and FLIP.

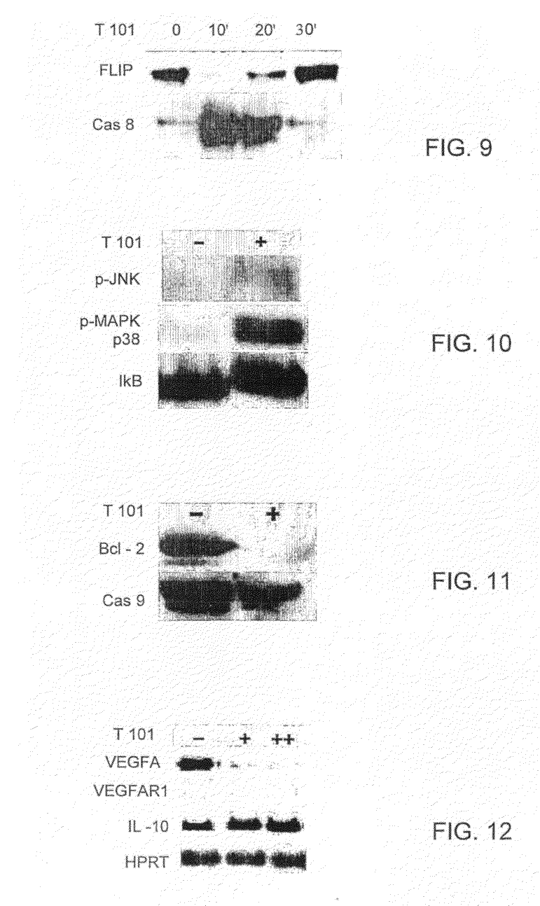

It may be seen in FIG. 9 that in untreated cells (time 0), a significant level of FLIP but very little caspase 8 are observed. After treatment with T101 the level of FLIP decreases and a concomitant increase of active caspase 8 is observed. At the same time, a decrease of the inactive caspase 8 is observed (not shown), which indicates that it is cleaved to yield the active caspase 8 species. It is seen that the changes in FLIP and caspase 8 are time-sensitive, with the greatest changes seen at the short time points (10′), after which their levels gradually return to baseline. Similar results were obtained with other cell lines as well. It was found that T101 was able to induce apoptosis in human breast cancer cells, human glioblastoma cells, human lung carcinoma cells, murine mammary gland carcinoma cells and mouse N2A neublastoma cells (not shown).

### Example 10

In order to determine the effect of T101 on various apoptotic signaling pathways, JNK and MAP kinase phosphorylation were measured.

In FIG. 10, it can be seen that T101 causes an increase in both JNK and MAPK phosphorylation 30 min after T101 application, while no change in the IκB protein level was detected. No change of IκB levels was detected at shorter time points as well. Therefore, it is assumed that the JNK pathway and MAP kinase pathway are activated by T101, while NF-κB remains unactivated. However, not only the caspase 8 pathway is activated upon T1/ST2 activation by T101, but also the caspase 9 dependent cascade is stimulated. Thus, in FIG. 11 it is seen that T101 causes a very pronounced degradation of the anti-apoptotic protein Bcl-2, accompanied by cleavage of the full-length caspase 9 protein that yields the active caspase 9 species.

These results strengthen the conclusion that T101 is an apoptosis-inducing peptide.

### Example 11

Angiogenesis is one of the key processes in the development of tumors. It was decided to study the effect of T1/ST2 receptor activation by T101 on the expression of various components of the angiogenic mechanism.

Mouse spleenocytes were challenged without or with increasing concentrations of T101. VEGFA, VEGFR1 and IL-10 expression were compared. VEGFA is a major proangiogenic cytokine, and VEGFR1 is a receptor for VEGFA. HPRT was included as a negative control.

It may be seen in FIG. 12 that T101 inhibits the expression of VEGFA and VEGFR1 in mouse spleenocytes. On the other hand, T101 enhanced in the same cells the expression of IL-10, which is a known anti-angiogenic interleukin. Upregulation of angiogenic factors has been demonstrated in endothelial cells that are mediated by TLR4. Since T1/ST2 activation is known to counteract TLR4 activity, it is anticipated that T101 would also cause downregulation of angiogenic factors in endothelial cells, in much the same way as in immune system cells.

It appears, therefore, that T101 has versatile effects, all having a common denominator of fighting the proliferation of transformed cells by both direct apoptosis and by inhibition of angiogenesis in the tumor.

### Example 12

A polyclonal antibody against T101 was raised and used for Immunological Histological Chemical (INC) studies in different human tissue sections. The results of the IHC analysis indicate that T101 is expressed exclusively in thymus sections. Other lymphatic lobes, spleen and liver sections did not bind the antibody. These results confirm the RT-PCR results, which suggested specific expression of T101 in the thymus. Specific staining in the thymus medulla and the thymus Hassall's corpuscles was seen, but no staining in the thymus cortex. The staining appears in the cell's cytoplasm as expected for a secreted peptide. The cells expressing T101 were identified as fibroblasts. In fetal thymus, more cells expressing T101 were seen in comparison with adult thymus (unpublished data).

1-20. (canceled)

21. A method for treating or preventing a disease involving a cell having a T1/ST2 receptor, in a subject in need thereof, comprising administering to the subject a therapeutically effective amount of an isolated polypeptide selected from the group consisting of
an isolated polypeptide comprising an amino acid sequence of SEQ. ID. NO: 1;
an isolated polypeptide comprising an amino acid sequence of SEQ. ID. NO: 1, in which one or more amino acid residues is added, deleted or replaced, without significantly affecting the biological characteristics of the modified molecule as compared to the unmodified molecule;
an isolated polypeptide comprising a partial contiguous sequence from SEQ. ID. NO: 1 that includes at least 8 amino acid residues, which contiguous sequence is included as a contiguous sequence in said SEQ. ID. NO: 1; and
an isolated polypeptide comprising a contiguous sequence of 13 amino acid residues beginning from the N-terminal of SEQ. ID. NO: 1,
wherein the disease involving a T1/ST2 receptor is selected from the group consisting of an acute cardiovascular disease, a chronic cardiovascular disease, idiopathic pulmonary fibrosis, Schistosomiasis and trauma.

22. The method according to claim 21, wherein the cardiovascular disease is selected from the group consisting of cardiac hypertrophy, myocardial infarction, stroke, atherosclerosis and heart failure.

23. The method according to claim 21, wherein the trauma is surgery-induced.

24. A method for treating or preventing a disease involving a cell having a T1/ST2 receptor, in a subject in need thereof, comprising administering to the subject a therapeutically effective amount of an antagonist of an isolated polypeptide selected from the group consisting of
an isolated polypeptide comprising an amino acid sequence of SEQ. ID. NO: 1;
an isolated polypeptide comprising an amino acid sequence of SEQ. ID. NO: 1, in which one or more amino acid residues is added, deleted or replaced, without significantly affecting the biological characteristics of the modified molecule as compared to the unmodified molecule;
an isolated polypeptide comprising a partial contiguous sequence from SEQ. ID. NO: 1 that includes at least 8 amino acid residues, which contiguous sequence is included as a contiguous sequence in said SEQ. ID. NO: 1; and
an isolated polypeptide comprising a contiguous sequence of 13 amino acid residues beginning from the N-terminal of SEQ. ID. NO: 1
wherein the disease involving a T1/ST2 receptor is selected from the group consisting of an acute cardiovascular disease, a chronic cardiovascular disease, idiopathic pulmonary fibrosis, Schistosomiasis and trauma.

25. A method for inhibiting the pathological effects of activated monocytes in a subject in need thereof, comprising treating the monocytes with an effective amount of an isolated polypeptide selected from the group consisting of
an isolated polypeptide comprising an amino acid sequence of SEQ. ID. NO: 1;
an isolated polypeptide comprising an amino acid sequence of SEQ. ID. NO: 1, in which one or more amino acid residues is added, deleted or replaced, without significantly affecting the biological characteristics of the modified molecule as compared to the unmodified molecule;
an isolated polypeptide comprising a partial contiguous sequence from SEQ. ID. NO: 1 that includes at least 8 amino acid residues, which contiguous sequence is included as a contiguous sequence in said SEQ. ID. NO: 1; and
an isolated polypeptide comprising a contiguous sequence of 13 amino acid residues beginning from the N-terminal of SEQ. ID. NO: 1.

26. The method of claim 25, wherein the pathological effects are selected from the group consisting of rejection of transplanted cells or tissues; autoimmune disease; arthritis; an inflammatory bowel disease; an endocrinopathy; a neurodegenerative disease; a vascular disease; rejection of allogeneic cells, tissues or organs; rejection of xenogeneic cells, tissues or organs; graft versus host disease; systemic or discoid lupus erythematosus; sclerosing cholangitis; autoimmune hepatitis; rheumatoid arthritis; psoriasis; psoriatic arthritis; ulcerative colitis; Crohn's disease; type 1 diabetes; Graves disease; multiple sclerosis; autistic spectrum disorder; Alzheimer's disease; amyotrophic lateral sclerosis (ALS); Parkinson's disease; Huntington's Disease; Guillain-Barre syndrome; myasthenia gravis; chronic idiopathic demyelinating disease (CID); autoimmune hearing loss; systemic vasculitis; and atherosclerosis.

27. The method of claim 26, wherein the pathological effect is the development of atherosclerotic plaques in blood vessels.

28. A method for protecting and preventing damage to the liver of a subject in need thereof, comprising administering to the subject a therapeutically effective amount of an isolated polypeptide selected from the group consisting of
an isolated polypeptide comprising an amino acid sequence of SEQ. ID. NO: 1;
an isolated polypeptide comprising an amino acid sequence of SEQ. ID. NO: 1, in which one or more amino acid residues is added, deleted or replaced, without significantly affecting the biological characteristics of the modified molecule as compared to the unmodified molecule;
an isolated polypeptide comprising a partial contiguous sequence from SEQ. ID. NO: 1 that includes at least 8 amino acid residues, which contiguous sequence is included as a contiguous sequence in said SEQ. ID. NO: 1; and
an isolated polypeptide comprising a contiguous sequence of 13 amino acid residues beginning from the N-terminal of SEQ. ID. NO: 1.

29. The method of claim 28, wherein the damage to the liver results from a pathological insult selected from the group consisting of damage caused by drugs and chemicals; liver inflammation; fatty liver (non alcoholic steatohepatitis (NASH)); hepatitis A; primary sclerosing cholangitis; Wilson's Disease; and alcohol related liver disease.

30. A method for decreasing blood levels of cholesterol in a subject in need thereof, comprising administering to the subject a therapeutically effective amount of an isolated polypeptide selected from the group consisting of
an isolated polypeptide comprising an amino acid sequence of SEQ. ID. NO: 1;
an isolated polypeptide comprising an amino acid sequence of SEQ. ID. NO: 1, in which one or more amino acid residues is added, deleted or replaced, without significantly affecting the biological characteristics of the modified molecule as compared to the unmodified molecule;
an isolated polypeptide comprising a partial contiguous sequence from SEQ. ID. NO: 1 that includes at least 8 amino acid residues, which contiguous sequence is included as a contiguous sequence in said SEQ. ID. NO: 1; and
an isolated polypeptide comprising a contiguous sequence of 13 amino acid residues beginning from the N-terminal of SEQ. ID. NO: 1.

31. The method of claim 30, for treating a disease associated with high blood levels of cholesterol.

32. The method of claim 31, wherein the disease is a cardiovascular disease.

33. The method of claim 32, wherein the cardiovascular disease is atherosclerosis.

34. A polypeptide having a sequence selected from the group consisting of
(SEQ ID NO: 12)
MDLSIRLSLACWELNQVSGAWGMSLKSHFKFMSDKQLISKA
VQRIFFSPSTLWGEK;
(SEQ ID NO: 13)
MSLKSHFKFMSDKQLISKAVQRIFFSPSTLWGEK;
(SEQ ID NO: 14)
MDLSIRLSLACWELNQVSGAWGMSLKSHFKFMSDKQLISKAVX₁X₂X₃
FFSPSTLWX₄X₅K;
and
(SEQ ID NO: 15)
MSLKSHFKFMSDKQLISKAVX1X2X3FFSPSTLWX₄X₅K;
where Xₙ is defined as follows;
X₁—Q,W
X₂—R,W
X₃—I,T
X₄—G,E
X₅—E,R.

35. A polynucleotide having a sequence selected from the group consisting of
(SEQ ID NO: 16)
ATGGACCTT TCCATCCGT CTGTCTCTT GCTTGCTGG GAGCTGAAC
CAGGTCTCT GGAGCATGG GGCATGAGC TTAAAATCC CATTTCAAG
TTCATGAGT GACAAGCAG CTAATTTCC AAAGCTGTG CAGCGGATA
TTTTTTTCT CCTTCAACC CTCTGGGGG GAAAAA TGA;
and
(SEQ ID NO: 17)
ATGAGC TTAAAATCC CATTTCAAG TTCATGAGT GACAAGCAG
CTAATTTCC AAAGCTGTG CAGCGGATA TTTTTTTCT CCTTCAACC
CTCTGGGGG GAAAAA TGA.

36. A polyclonal antibody which binds T101.

37. A pharmaceutical composition comprising the antibody of claim 36 for use in treating or preventing a disease involving a cell having a T1/ST2 receptor, in a subject in need.

38. The pharmaceutical composition of claim 37, wherein the disease involving a T1/ST2 receptor is selected from the group consisting of an acute cardiovascular disease, a chronic cardiovascular disease, idiopathic pulmonary fibrosis, Schistosomiasis and trauma.
